(19) United States
(12) Patent Application Publication
(10) Pub. No.: US 2023/0287494 A1
(43) Pub. Date: Sep. 14, 2023

(54) METHODS FOR SCREENING EMBRYOS

(71) Applicant: Shahryar K. KAVOUSSI, Austin, TX (US)

(72) Inventor: Shahryar K. KAVOUSSI, Austin, TX (US)

(21) Appl. No.: 18/019,045

(22) PCT Filed: Jul. 30, 2021
(86) PCT No.: PCT/US2021/071065

§ 371 Date: Jan. 31, 2023

Related U.S. Application Data
(60) Provisional application No. 63/165,221, filed on Mar. 24, 2021.
(60) Provisional application No. 63/059,761, filed on Jul. 31, 2020.

(51) Int. Cl. C12Q 1/6876; C12Q 1/6869

(52) U.S. Cl. CPC C12Q 1/6876; C12Q 1/6869; C12Q 2600/158

(57) ABSTRACT

Methods of assessing obstetric outcomes of embryos based on the expression of pappalysin-1 (PAPPA), e.g., as measured in a biopsy from an embryo at the blastocyst stage, blastocoel fluid, or embryo culture conditioned media, are provided.

This application claims the benefit of U.S. Provisional Patent Application No. 63/059,761, filed Jul. 31, 2020, and United States Provisional Patent Application No. 63/165,221, filed Mar. 24, 2021, the entirety of which are incorporated herein by reference.

### BACKGROUND OF THE INVENTION

Pursuant to 37 C.F.R. 1.821(c), a sequence listing is submitted herewith as an ASCII compliant text file named “SHAHP0002WO_ST25.txt”, created on Jul. 30, 2021 and having a size of ˜1 kilobytes. The content of the aforementioned file is hereby incorporated by reference in its entirety.

### 1. Field of the Invention

The present invention relates generally to the field of molecular biology and medicine. More particularly, it concerns methods of screening embryos for viability and improved obstetric outcomes.

### 2. Description of Related Art

Although in vitro fertilization (IVF) has been successfully used for several decades, adverse obstetric outcomes including spontaneous miscarriage continue to be a risk for IVF procedures and patients. A need exists for early detection and prevention of adverse maternal and fetal prenatal outcomes. While some genetic screening, such as screening for aneuploidy, have been very helpful, such risks have by no means been eliminated. Additional screening techniques are needed to improve outcomes for IVF cases, including methods that can be used to predict such outcomes at the embryonic stage.

### SUMMARY OF THE INVENTION

The present invention overcomes limitations in the art, in some aspects, by providing improved methods for identifying embryos with decreased risk of adverse obstetric outcomes. For example, in some embodiments, decreased expression of Pappalysin-1 (PAPPA, also called pregnancy-associated plasma protein-A or PAPP-A) in an embryo tissue such as trophectoderm cells or syncytiotrophoblast cells, or in the blastocoel fluid, can indicate an increased risk of an adverse obstetric outcome (e.g., resulting in loss of the embryo during a pregnancy). In some aspects, methods of selecting embryos for use in an in vitro fertilization (IVF) procedure are provided. In some aspects, the methods provided herein can be used for preimplantation prenatal screening (PPS).

In some aspects, methods of in vitro detection of PAPPA in embryonic biopsies, blastocoel fluid, and/or embryo culture conditioned medium of blastocyst stage preimplantation human embryos are provided. By comparison of the degree of mRNA expression or protein expression of PAPPA by preimplantation blastocyst stage embryos, an additional level of embryo selection, in addition to euploid status, can be provided in to choose embryo(s) for transfer to the uterus in an in vitro fertilization patient.

An aspect of the present disclosure relates to an in vitro method of screening an embryo, comprising: (i) obtaining in vitro an embryo at the blastocyst stage of development, and (ii) measuring the expression of pregnancy-associated plasma protein-A (PAPPA) in one or more embryo cells, in the blastocoel fluid, and/or in embryo culture fluid that has been used to store or culture the embryo; wherein a decreased expression of PAPPA below a control level indicates an increased risk of an adverse obstetric outcome if the embryo is implanted into a mammalian subject. In some embodiments, the expression of PAPPA is measured in the blastocoel fluid, in the embryo culture fluid, or in one or more embryo cells. The one or more embryo cells may comprise or consist of one or more trophoblast or trophectoderm cells. The one or more trophoblast cells may comprise or consist of syncytiotrophoblast cells. The one or more embryo cells may comprise or consist of one or more inner cell mass cells. In some embodiments, the adverse obstetric outcome is abnormal placentation, spontaneous miscarriage, small for gestational age (SGA), intrauterine growth restriction (IUGR), intrauterine fetal demise (IUFD), or pre-eclampsia. In some embodiments, the measuring is performed via detecting or measuring mRNA that encodes PAPPA. For example, the measuring may be performed via Northern blot analysis, nuclease protection assays (NPA), in situ hybridization, reverse transcription-polymerase chain reaction (RT-PCR), or next-generation mRNA sequencing (mRNA-Seq). In some embodiments, the reverse transcription-polymerase chain reaction (RT-PCR) is further defined as a reverse transcription quantitative PCR (RT-qPCR) or a semi-quantitative PCR. In some embodiments, the measuring is performed via detecting or measuring PAPPA protein is a Western blot, high-performance liquid chromatography (HPLC), liquid chromatography-mass spectrometry (LC/MS), enzyme-linked immunosorbent assay (ELISA), protein immunostaining, or electrochemiluminescence immunoassay (ECLIA). In some embodiments, the measuring is performed via ELISA. The method may further comprise testing the embryo for aneuploidies (PGT-A). The method may further comprise selecting an embryo for implantation into the mammalian subject. In some embodiments, the embryo is implanted into the mammalian subject as part of an in vitro fertilization (IVF) method. In some embodiments, the embryo has been generated from an oocyte and/or sperm obtained from a donor. The donor is the mammalian subject, such as a human. In some embodiments, the donor is a first human subject, and wherein the mammalian subject is a second human subject. In some embodiments, the mammalian subject is a cow, sheep, horse, dog, cat, lion, panther, ferret, goat, or pig. The mammalian subject may be a domesticated animal. In some embodiments, the mammalian subject is an endangered species. In some embodiments, the mammalian subject is a human. The method may further comprise performing additional genetic testing. The additional genetic testing may comprise or consist of testing for the presence of a genetic disease in the embryo. In some embodiments, the embryo is further defined as a human embryo, and wherein the genetic disease is Huntington's disease, sickle cell anemia, muscular dystrophy, cystic fibrosis (CF), a BRCA1 mutation, a BRCA2 mutation, fragile-X syndrome, or Tay-Sachs disease.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01% (w/w). Most preferred is a composition in which no amount of the specified component can be detected with standard analytical methods.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The present disclosure, in some aspects, provides improved methods for selecting embryos for implantation (e.g., for an in vitro fertilization (IVF) procedure) that have improved viability or a reduced risk of an adverse obstetric outcome. In some aspects, decreased expression of PAPPA in an embryonic tissue, the blastocoel fluid, or embryo culture media as measured by either mRNA or protein levels can indicate an increased risk of an adverse obstetric outcome (e.g., abnormal placentation, increased risk of spontaneous miscarriage, small for gestational age (SGA), intrauterine growth restriction (IUGR), intrauterine fetal demise (IUFD), or pre-eclampsia).

### I. PAPPA

PAPPA (also called pappalysin-1, pregnancy-associated plasma protein-A or PAPP-A) is a serine protease that is expressed by cells including human fibroblasts. PAPPA has been shown to be as a pregnancy-dependent oncogene. Transgenic expression of PAPP-A in the mouse mammary gland during pregnancy and involution promotes the deposition of collagen, and lactation can protect against these effects (Takabatake et al., 2016). PAPPA mRNA levels in cumulus cells obtained from unfertilized oocytes has been associated with oocyte developmental competence (Kordus et al., 2019). PAPPA is a metalloproteinase which selectively cleaves IGFBP-4 and IGFBP-5, resulting in release of IGF. Cleavage of IGFBP-4 can be enhanced by the presence of IGF, whereas cleavage of IGFBP-5 may be slightly inhibited by the presence of IGF. PAPPA can play a role in bone formation, inflammation, wound healing and female fertility. PAPPA has been sequenced in a variety of mammalian organisms, including in humans (e.g., Homo sapiens pappalysin-1, Gene ID: 5069).

In the realm of IVF-conceived pregnancies, there are conflicting data regarding first trimester maternal serum PAPP-A levels as compared with pregnancies achieved without IVF. Some data suggest no difference in PAPP-A levels in pregnancies resulting from fresh embryo transfer (ET), frozen embryo transfer (FET), or spontaneous conception (Cavoretto et al, 2016), and other studies have shown lower first trimester maternal serum PAPP-A levels in pregnancies conceived as a result of either fresh ET or FET as compared with spontaneous pregnancies (Liao et al, 2001; Hui et al, 2005; Tul et al, 2006; Amor et al, 2009; Gjerris et al, 2009; Matilainen et al, 2011; Gjerris et al, 2012). One study showed no correlation between blastocyst morphology parameters and first trimester maternal serum PAPP-A levels in ongoing pregnancies (Pėrennec et al, 2020). Some data to suggest that peak E2 level on the day of trigger is not associated with low maternal serum PAPP-A levels in pregnancies that result from fresh ET (Dunne et al., 2017), although another publication demonstrated lower maternal serum PAPP-A levels in IVF and ICSI pregnancies compared with non-IVF and ICSI pregnancies. In the latter study, a correlation was found between peak E2 level at triggering and low first trimester maternal serum PAPP-A in IVF pregnancies (Giorgetti et al., 2013). Placental volume of pregnancies achieved via FET have been shown to be greater than that of pregnancies resulting from fresh ET and spontaneous pregnancy, with a positive correlation observed between placental volume and first trimester maternal serum PAPP-A levels (Choux et al, 2019).

In contrast to this previous work, methods are provided herein for selecting embryos for implantation (e.g., for an in vitro fertilization (IVF) procedure) that have improved viability and/or reduced risk of an adverse obstetric outcome, based on either (i) selecting embryos that display increased expression of PAPPA in an embryonic tissue, the blastocoel fluid, or embryo culture media, and/or (ii) excluding embryos that display decreased expression of PAPPA in an embryonic tissue, the blastocoel fluid, or embryo culture media. As shown in the examples, frozen euploid blastocyst stage embryos that had been vitrified after trophectoderm biopsy were analyzed for PAPPA expression.

### II. PAPPA Detection Methods

A variety of techniques can be used to detect mRNA encoding PAPPA or PAPPA protein. The mRNA encoding PAPPA or PAPPA protein may be measured in more embryo cells (e.g., trophoblast or trophectoderm cells, syncytiotrophoblast cells, or inner cell mass cells) in the blastocoel fluid, and/or in embryo culture fluid that has been used to store or culture the embryo.

A variety of methods may be used to detect or measure mRNA that encodes PAPPA. For example, in various embodiments, the method may be Northern blot analysis, nuclease protection assays (NPA), in situ hybridization, reverse transcription-polymerase chain reaction (RT-PCR), or next-generation mRNA sequencing (mRNA-Seq).

In some embodiments, RT-PCR is performed to measure PAPPA mRNA. For example, in some embodiments, the following method may be used. RNA can be extracted with TRIzol from a sample. The samples can be centrifuged for 10 min at 11,200 g and the resulting RNA pellet was washed once with 70% ethanol and resuspended in 40 μl of diethyl pyrocarbonate-treated water. cDNA can be synthesized, e.g., using a Takara cDNA synthesis kit (Takara Bio, Inc., Otsu, Japan) following the manufacturer's protocol. qPCR can be conducted using SYBR. In some embodiments, the following primers can be used for amplification of PAPPA: forward primer GTCATCTTTGCCTGGAAGGGAGAA (SEQ ID NO:1) and reverse primer AGGGCTGTTCAACATCAGGATGAC (SEQ ID NO:2), e.g., at about 56° C. In some embodiments, the reverse transcription-polymerase chain reaction (RT-PCR) is a reverse transcription quantitative PCR (RT-qPCR) methodology or a semi-quantitative PCR methodology. Semi-quantitative PCR methods are known in the art and include those described, e.g., in Chen et al., 1999.

In some embodiments, next-generation sequencing is used to measure PAPPA mRNA via RNA-seq (RNA-sequencing). RNA-seq methods using next-generation sequencing can be used to quantify expression of genes (e.g., Mortazavi et al., 2008; Trapnell et al., 2010). Next-generation sequencing methods that may be used include: sequencing methods that identifying DNA bases based on the emission of a unique fluorescent signal as nucleic acids are added to a nucleic acid chain (e.g., by Ilumina (Solexa)), pyrosequencing methods (e.g., 454 sequencing), and detection of nucleic acid incorporation by detection of hydrogen ions with a semiconductor (e.g., Ion Torrent methods). Next generation sequencing includes massively parallel signature sequencing, polony sequencing, cPAS sequencing, SOLiD sequencing, DNA nanoball sequencing, and SMRT PacBio single molecule real time sequencing (e.g., by Pacific Bioscience).

Additional methods that can be used to measure PAPPA mRNA include quantitative real-time RT-PCR. Real-time RT-PCR has been successfully use in a wide variety of fields for some time. This method can be used for measuring mRNA levels of in vivo low copy number targets of interest. Benefits of this procedure over other methods for measuring RNA include its sensitivity, large dynamic range, the potential for high throughout as well as accurate quantification (Huggett, et al., 2005).

In some embodiments, PAPPA protein levels are measured from in more embryo cells (e.g., trophoblast or trophectoderm cells, syncytiotrophoblast cells, or inner cell mass cells) in the blastocoel fluid, and/or in embryo culture fluid that has been used to store or culture the embryo. A variety of methods for detection of PAPPA protein may be used. For example, methods that may be used include: Western blot, high-performance liquid chromatography (HPLC), liquid chromatography-mass spectrometry (LC/MS), enzyme-linked immunosorbent assay (ELISA), protein immunostaining, or electrochemiluminescence immunoassay “ECLIA” (Elecsys). In some embodiments, microfluidics may be used to quantify the PAPPA protein.

In some aspects, the level of expression of PAPPA is compared to a control level or normal level of expression. In some embodiments, the control level of normal level of expression can be established in experiments and quantified at the embryonic level, for example with using culture medium as a negative control. Decreased expression of PAPPA can be indicative of an adverse obstetric outcome (e.g., is abnormal placentation, spontaneous miscarriage, small for gestational age (SGA), intrauterine growth restriction (IUGR), intrauterine fetal demise (IUFD), or pre-eclampsia). Thus, the methods provided herein can be used in selecting an embryo for implantation to increase the chances of a healthy, live birth as part of an IVF protocol.

### III. Embryonic Tissues for PAPPA Testing

It is anticipated that a variety of embryonic tissues at the blastocyst stage of development, the blastocoel fluid, and/or the embryo culture media, can be used to measure PAPPA expression in a method as disclosed here, e.g., to identify embryos for use in an IVF procedure. In some embodiments, a tissue sample or biopsy is obtained the embryo is at day 3, 4, 5, 6, of 7 of development; for example, a biopsy of one cell from day 3 may be obtained, or a biopsy of one or multiple cells (e.g., 1, 2, 3, 4 cells) from a day 5, day 6, or day 7 embryo may be obtained. The embryonic tissue may comprise or consist of trophoblasts, such as syncytiotrophoblast cells.

Embryonic tissues at the blastocyst stage of development include trophoblasts, the blastocyst cavity (blastocoele), and inner cell mass (embryoblast). Trophoblasts (trophectoderm cells) form the outer layer of the blastocyst and are normally observed within four to six days after fertilization in humans. Some trophectoderm cells can differentiate into become extraembryonic structures, and trophectoderm cells do not directly contribute to the embryo. Syncytiotrophoblast cells are a type of trophoblast cell that form the epithelial covering of vascular embryonic placental villi, which invade the wall of the uterus and are involved in obtaining nutrients from the mother.

In some embodiments, one or more cells from the inner cell mass can be used as the tissue biopsy for measuring PAPPA levels. In some instances, the embryonic tissue biopsy is not the inner cell mass, since these cells will form the developing mammalian subject; nonetheless, a recent study supports the idea that inner cell mass cells from a blastocyst may be obtained without adversely impairing the developing embryo (Scott et al., 2013).

In some embodiments, a tissue biopsy is obtained from the embryo to detect and measure expression of PAPPA. For example, in order to access embryonic cells for biopsy, which may be trophectoderm (TE) and/or inner cell mass (ICM) cells, laser pulses can be applied to the zona pellucidae which surround a blastocyst stage embryo and then the laser is applied to the junctions between embryonic cells in order to obtain a biopsy. The biopsied embryonic cells can then be removed (e.g., by pipette), and can be placed into a buffer solution. PAPPA can then be measured as described herein, e.g., by detecting or quantifying PAPPA mRNA or protein. After obtaining the blastocyst biopsy, the embryo may collapse, resulting in blastocoel fluid being extruded out into the surrounding medium.

Embryo culture medium containing blastocoel fluid (ECB), also referred to as blastocoel fluid conditioned medium (BFCM) can also be obtained and used to test for PAPPA expression. A variety of methods can be used to obtain a sample of blastocoel fluid. Methods for obtaining ECB are described, e.g., in Li et al., 2018 or Stigliani, 2014. In some embodiments, ECB can be obtained via the following method. An infrared laser can be used to lase a small breech in the zone pellucida (ZP) far away from the inner cell mass to release the blastocoels fluid into the culture medium. The released blastocoel fluid mixed with culture media (e.g., about 25 μl) can be transferred to RNase-DNase-free tubes for subsequent analysis (e.g., PCR). To prevent media contamination, different Pasteur pipettes can be used for each sample.

Trophectoderm cells can be obtained by a variety of techniques known in the art. For example, a laser or biopsy pipette can be used to obtain the trophectoderm cells. In some embodiments, trophectoderm cells were encouraged to herniate from the zona by applying gentle suction with the biopsy pipette. One or multiple trophectoderm cells (e.g., 1, 2, 3, 4, or 5 cells) may be dissected from a blastocyst using a laser (e.g., four laser pulses of 3 seconds in duration). The biopsied cells can be placed immediately in RNase-DNase-free tubes for further analysis of PAPPA expression as described herein (e.g., using PCR etc.).

In some embodiments, embryo culture medium containing blastocoel fluid (ECB) is used. Blastocoel fluid conditioned media, which is routinely discarded, can be collected and saved (e.g., post biopsy) from a Day 5, Day 6, or Day 7 blastocyst stage mammalian embryo (such as a human embryo). In some embodiments, the biopsy is obtained from embryos that are being analyzed for possible implantation in a patient undergoing IVF, for example along with a preimplantation genetic testing for aneuploidy. Additional methods for obtaining blastocoel fluid conditioned media that may be used include, e.g., Chosed et al., 2019; Vera-Rodriguez et al., 2018; Rule et al., 2018; and Xu et al., 2016.

### IV. In Vitro Fertilization (IVF)

Measuring expression of PAPPA in an embryo at the blastocyst stage to predict an obstetric outcome may be performed as part of an IVF procedure. In vitro fertilization includes a variety of techniques for assisting with the conception and birth of a child. In some preferred embodiments, the IVF procedure is for a human patient. Nonetheless, techniques disclosed herein can be applied to a wide variety of mammals, including domesticated animals, including cows, horses, dogs, cats, sheep, goats, or pigs, or endangered species, such as various lions and panthers.

Generally, during IVF mature eggs are collected from ovaries from a mammalian and fertilized by sperm in a lab. The fertilized egg (embryo) or eggs (embryos) are then transferred to a uterus. One full cycle of IVF takes about three weeks. These steps can be separated into different parts, if desired. The IVF procedure may involve intracytoplasmic sperm injection (ICSI) (e.g., Neri et al., 2014). IVF has been used in various forms since its introduction in 1978.

A variety of additional tests may be performed on the embryo prior to implantation in an IVF procedure. For example, blastocoel fluid or a tissue biopsy from the embryo (e.g., trophectoderm cells that are also used to assess PAPPA expression as described herein) can be analyzed with fluorescence in situ hybridization (FISH), array comparative genomic hybridization (aCGH), single-nucleotide polymorphism (SNP) arrays, multiplex quantitative PCR or next generation sequencing (NGS) to test for aneuploidy, determine the chromosomal status of the embryo, and/or to facilitate selection of desired embryos for implantation.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Analysis of PAPPA to Select Embryos for In Vitro Fertilization (IVF) Methods

Analysis of PAPPA to Select Embryos for In Vitro Fertilization (IVF) can be performed via the following methods. Prior to and/or at the time of blastocyst-stage embryo biopsy, blastocoel fluid collection and/or embryo culture medium is collected at a volume of 25 μL and is snap frozen prior to shipment to the reference research laboratory. In order to access embryonic cells for biopsy, which may be trophectoderm and/or inner cell mass cells, laser pulses are first applied to the zona pellucidae which surround the blastocyst stage embryos and then laser is applied to the junctions between embryonic cells in order to perform biopsy. The biopsied embryonic cells are then removed by pipette and placed into buffer for subsequent PAPP-A testing, e.g., at a research laboratory. At completion of the blastocyst biopsy, the embryo collapses, resulting in blastocoel fluid being extruded out into the surrounding medium.

Blastocoel fluid conditioned media, which is routinely discarded, is collected and saved post biopsy from Day 5 or Day 6 blastocyst stage human embryos obtained from patients undergoing IVF with preimplantation genetic testing for aneuploidy. The blastocyst fluid-conditioned culture media with a volume of approximately 25 μL is snap frozen prior to shipment to the reference research laboratory.

Embryo culture conditioned medium (spent medium) is collected from the patient's embryo culture medium. Approximately 25 μL of the spent medium is snap frozen prior to shipment to the reference research laboratory.

Biopsied embryos are cryopreserved pending outcome of the sequencing results as some cells from the biopsy will be sent for PGT-A analysis via Next-Generation Sequencing (NGS) at a commercial reference laboratory. Cells from embryonic cell biopsies, the blastocoel fluid, and the embryo culture fluid will be sent to a research laboratory and will be tested for PAPP-A protein and PAPP-A mRNA.

### Example 2

### Expression of Pregnancy-Associated Plasma Protein-A (PAPP-A) in Human Blastocoel Fluid-Conditioned Media

Methods: Blastocoelic fluid conditioned media (BFCM) was obtained from blastocyst stage embryos following standard, routine controlled ovarian stimulation and subsequent IVF processes. Female patients had undergone their planned routine IVF cases, which consisted of controlled ovarian stimulation with exogenous gonadotropins, the use of gonadotropin hormone antagonist for suppression of luteinizing hormone prior to trigger with leuprolide acetate and/or recombinant human Chorionic Gonadotropin (hCG) for final oocyte maturation prior to transvaginal ultrasound-guided oocyte retrieval 35 hours later. Oocytes were isolated and intracytoplasmic sperm injection was performed to achieve in vitro fertilization, with culture of embryos to the blastocyst stage of embryonic development by Day 5 and Day 6 of embryo culture. Good quality blastocysts were considered to be those with a grade of 2BB or higher, in accordance with Gardner and Schoolcraft's grading system for blastocysts (Gardner et al, 2000). All 80 of the blastocysts in this study had undergone trophectoderm biopsy for preimplantation genetic testing for aneuploidy (PGT-A) prior to blastocyst vitrification and BFCM collection.

Per routine embryology laboratory protocol, each blastocyst was placed in a 20 μL medium drop under oil, laser pulse was used to open the cellular junctions between trophectoderm cells and trophectoderm biopsy was performed so that the biopsied cells could be sent to a reference genetics laboratory for PGT-A via next generation sequencing. As the blastocyst collapsed, blastocoel fluid was extruded into the drop of medium and the blastocyst was removed from the medium drop for subsequent blastocyst vitrification. The medium drop containing BF was collected and mixed via pipetting. Each BFCM sample was subsequently stored at −20° C. for further analysis.

RT-qPCR was performed for PAPP-A. Blastocoel fluid-conditioned media from individual euploid pre-implantation embryos were assessed for RNA content using a RNA 6000 Pico Kit with an Agilent 2100 Bioanalyzer. Individual samples were treated with RNase-free DNase 1 for 30 minutes at 37° C. followed by heat inactivation. Samples were then subjected to cDNA synthesis with a High-Capacity cDNA Reverse Transcription Kit (Applied Biosystems) per manufacturer's instructions. cDNA quantity was then determined with a High Sensitivity DNA Kit with an Agilent 2100 Bioanalyzer. cDNA was then combined with 2×TaqMan Master Mix, 20×Gene Expression Assay (GAPDH and PAPP-A specific) and RNase-free water per manufacturer's instructions (Applied Biosystems). Duplicate reactions for each sample were run for each gene of interest using a 7500 Fast Real-Time PCR System (Applied Biosystems, USA) at 50° C. for 2 minutes, 95° C. for 20 seconds, followed by 40 cycles of 95° C. for 3 seconds and 60° C. for 30 seconds.

IRB exemption was obtained by St. David's Institutional Review Board due to the de-identified nature of the data as well as the use of BFCM that routinely would be discarded at the time of collapsing blastocysts for vitrification.

Results: A sample size of 80 trophectoderm-tested euploid Day 5 good quality blastocysts from 36 patients that underwent IVF/ICSI/PGT-A/freeze-all had BFCM analyzed for PAPP-A mRNA expression. Patient and embryonic characteristics are listed in Table 1. PAPP-A mRNA was detected in 45 of 80 BFCM samples (56.3%), with varying levels of expression across samples (Table 2). Mean age in years for the subjects with at least one BFCM sample with PAPP-A mRNA detected (n=26) and those with no BFCM samples with detectable PAPP-A mRNA (n=10) were 36.1 years and 36.4 years, respectively. Of the 45 blastocysts that had PAPP-A mRNA detected in their BFCM samples, 36 blastocysts (80%) had trophectoderm grading of A and 9 blastocysts had trophectoderm grading of B; of the 35 blastocysts that had no detectable PAPP-A mRNA in their BFCM samples, 28 (80%) had trophectoderm grading of A and 7 blastocysts had trophectoderm grading of B. Pregnancy outcome data of embryo transfers (n=28) are shown in Table 2.

TABLE 1
Characteristics and variables, including demographics
and outcomes for study subjects.

| | +PAPP-A | −No PAPP-A |
|---|---|---|
| | in BFCM | in BFCM |
| | (n = 26) | (n = 10) |
| # of subjects | 26 | 10 |
| Female age (mean) in years | 36.1 | 36.4 |
| Number of blastocysts | 45 | 35 |
| Trophectoderm Grade | | |
| A | 36 | 28 |
| B | 9 | 7 |

TABLE 2
Pregnancy results of euploid blastocyst transfer, +PAPP-A
expression in BFCM vs −PAPP-A expression in BFCM

| | +PAPP-A | No PAPP-A | |
|---|---|---|---|
| | (n = 13) | (n = 15) | P- value* |
| Pregnant | 9 | 10 | 1 |
| Ongoing pregnancy | 5 | 6 | 1 |
| SAB | 4 | 4 | 1 |
| Not pregnant | 4 | 5 | 1 |

*Fisher's Exact test

TABLE 3
Pregnancy after embryo transfers

| | | | | | | | | PAPP-A | |
|---|---|---|---|---|---|---|---|---|---|
| Sample | Patient | Female | Day 5 | Embryo | PGT | | PAPP-A | Expression | |
| No. | No | Age | or 6 | Grade | result | Pregnancy | result | (fold change) | Comment |
| Control | | | | | | | none | No Expression | |
| B5 | 1 | 38 | 5 | 3AA | Normal | YES | none | No Expression | SAB |
| B6 | 1 | 38 | 5 | 3AA | Normal | YES | none | No Expression | SAB |
| B7 | 2 | 34 | 5 | 4AB | Normal | YES | none | No Expression | SAB |
| B9 | 3 | 41 | 5 | 3AA | Normal | YES | none | No Expression | Ongoing |
| | | | | | | | | | pregnancy |
| B10 | 3 | 41 | 5 | 2AA | Normal | | none | No Expression | |
| B12 | 4 | 35 | 5 | 4AA | Normal | YES | none | No Expression | Ongoing |
| | | | | | | | | | pregnancy |
| B14 | 4 | 35 | 5 | 3AA | Normal | | none | No Expression | |
| B15 | 4 | 35 | 5 | 3AA | Normal | | none | No Expression | |
| B17 | 5 | 39 | 5 | 4AA | Normal | NO | none | No Expression | |
| B18 | 5 | 39 | 5 | 4AA | Normal | YES | YES | 7987.377606 | Ongoing |
| | | | | | | | | | pregnancy |
| B22 | 6 | 39 | 5 | 4AA | Normal | YES | none | No Expression | Ongoing |
| | | | | | | | | | pregnancy |
| B23 | 6 | 39 | 5 | 4AB | Normal | | YES | 493.3324972 | |
| B28 | 7 | 36 | 5 | 4AA | Normal | YES | YES | 4599.994799 | Ongoing |
| | | | | | | | | | pregnancy |
| B29 | 7 | 36 | 5 | 4AA | Normal | | none | No Expression | |
| B31 | 7 | 36 | 5 | 3AB | Normal | | none | No Expression | |
| B32 | 8 | 33 | 5 | 4AA | Normal | YES | YES | 7411.337706 | Ongoing |
| | | | | | | | | | pregnancy |
| B33 | 8 | 33 | 5 | 4AA | Normal | | none | No Expression | |
| B36 | 8 | 33 | 5 | 2AA | Normal | | none | No Expression | |
| B41 | 9 | 33 | 5 | 3AA | Normal | | YES | 1597.409241 | |
| B42 | 10 | 35 | 5 | 4AA | Normal | | none | No Expression | |
| B44 | 10 | 35 | 5 | 4AA | Normal | | YES | 2145.911468 | |
| B45 | 10 | 35 | 5 | 3AA | Normal | | YES | 601.9724504 | |
| B46 | 10 | 35 | 5 | 3AA | Normal | | YES | 15251.45034 | |
| B48 | 11 | 38 | 5 | 4AA | Normal | YES | YES | 4.352961737 | SAB |
| B49 | 11 | 38 | 5 | 3AA | Normal | YES | None | No Expression | Ongoing |
| | | | | | | | | | pregnancy |
| B52 | 12 | 35 | 5 | 3AA | Normal | YES | YES | 3530.484869 | SAB |
| B53 | 12 | 35 | 5 | 3AA | Normal | | None | No Expression | |
| B58 | 13 | 35 | 5 | 4AA | Normal | NO | None | No Expression | |
| B61 | 13 | 35 | 5 | 3AB | Normal | | None | No Expression | |
| B64 | 14 | 35 | 6 | 4AA | Normal | YES | YES | 2911.430726 | Ongoing |
| | | | | | | | | | pregnancy |
| B65 | 15 | 42 | 5 | 3AA | Normal | NO | YES | 1.952757196 | |
| B70 | 16 | 41 | 5 | 2AB | Normal | YES | YES | 7442.195515 | Ongoing |
| | | | | | | | | | pregnancy |
| B72 | 17 | 36 | 5 | 3AA | Normal | NO | none | No Expression | |
| B80 | 18 | 39 | 5 | 2AB | Normal | YES | none | No Expression | SAB |
| B81 | 19 | 38 | 5 | 3AA | Normal | YES | none | No Expression | Ongoing |
| | | | | | | | | | pregnancy |
| B83 | 19 | 38 | 6 | 4AB | Normal | | Yes | 881.8657216 | |
| B86 | 20 | 39 | 5 | 2AA | Normal | | none | No Expression | Ongoing |
| | | | | | | YES | | | pregnancy |
| B87 | 20 | 39 | 5 | 2AA | Normal | | Yes | 3189.059254 | |
| B91 | 21 | 36 | 5 | 3AB | Normal | NO | none | No Expression | |
| B93 | 22 | 38 | 5 | 4AA | Normal | | Yes | 146.6262909 | Chemical |
| | | | | | | YES | | | pregnancy |
| B94 | 22 | 38 | 5 | 3AA | Normal | | Yes | 5.979396995 | |
| B98 | 23 | 36 | 5 | 4AA | Normal | NO | Yes | 17.27363733 | |
| B99 | 23 | 36 | 6 | 4AA | Normal | | Yes | 139.9725386 | |
| B100 | 23 | 36 | 6 | 3AA | Normal | | none | No Expression | |
| B101 | 23 | 36 | 6 | 3AB | Normal | | Yes | 209.382927 | |
| B102 | 24 | 37 | 5 | 4AA | Normal | | Yes | 124.4135665 | Chemical |
| | | | | | | YES | | | pregnancy |
| B103 | 24 | 37 | 5 | 4AA | Normal | NO | Yes | 1.301792944 | |
| B104 | 24 | 37 | 5 | 3AA | Normal | | Yes | 227.7015023 | |
| B105 | 24 | 37 | 5 | 3AA | Normal | | none | No Expression | |
| B108 | 24 | 37 | 5 | 2AA | Normal | | none | No Expression | |
| B114 | 24 | 37 | 6 | 3AB | Normal | | Yes | 115.200247 | |
| B116 | 25 | 33 | 5 | 4AA | Normal | NO | Yes | 1 | |
| B117 | 25 | 33 | 5 | 3AB | Normal | | none | No Expression | |
| B120 | 26 | 38 | 6 | 3AA | Normal | | none | No Expression | |
| B122 | 27 | 32 | 5 | 3AA | Normal | NO | none | No Expression | |
| B123 | 27 | 32 | 5 | 3AB | Normal | | none | No Expression | |
| B126 | 28 | 29 | 5 | 3AA | Normal | | Yes | 9998.513201 | |
| B127 | 28 | 29 | 5 | 3AB | Normal | | Yes | 6044.879494 | |
| B137 | 29 | 36 | 5 | 3AA | Normal | | Yes | 6100.068088 | |
| B139 | 29 | 36 | 5 | 3AA | Normal | | Yes | 537.6657218 | |
| B140 | 29 | 36 | 5 | 3AA | Normal | | Yes | 7442.696186 | |
| B142 | 29 | 36 | 5 | 3AB | Normal | | Yes | 714.3887363 | |
| B143 | 29 | 36 | 5 | 3AB | Normal | | Yes | 584.5850685 | |
| B146 | 30 | 40 | 5 | 4AA | Normal | | Yes | 250326.8627 | |
| B148 | 30 | 40 | 5 | 2AA | Normal | | Yes | 3084.441476 | |
| B153 | 31 | 34 | 6 | 3AA | Normal | | Yes | 486.7281556 | |
| B154 | 32 | 35 | 5 | 3AA | Normal | | Yes | 73.12961395 | |
| B155 | 32 | 35 | 5 | 3AA | Normal | | none | No Expression | |
| B156 | 32 | 35 | 5 | 2AA | Normal | | Yes | 168.9166902 | |
| B158 | 33 | 30 | 6 | 3AA | Normal | | Yes | 60.8071081 | |
| B160 | 34 | 36 | 5 | 4AA | Normal | | Yes | 4619.970978 | |
| B162 | 34 | 36 | 5 | 3AA | Normal | | Yes | 6.082515424 | |
| B163 | 34 | 36 | 5 | 3AA | Normal | | Yes | 596.4130742 | |
| B165 | 34 | 36 | 5 | 2AA | Normal | | none | No Expression | |
| B166 | 34 | 36 | 6 | 4AA | Normal | | Yes | 16295.29753 | |
| B172 | 35 | 35 | 5 | 3AA | Normal | | none | No Expression | |
| B173 | 35 | 35 | 5 | 3AA | Normal | | none | No Expression | |
| B175 | 35 | 35 | 5 | 3AB | Normal | | Yes | 573.8657212 | |
| B178 | 36 | 36 | 5 | 4AA | Normal | | Yes | 13844.92281 | |
| B179 | 36 | 36 | 5 | 4AA | Normal | | Yes | 2957.309228 | |

Meaning of Fold Change Ratio: Expression in B18 is 16.19 times more than in B23, and Expression in B18 is 16.19/9.23 times more than in B28.

### Definitions of SAB and SAB/Chemical in Table 2:

SAB=Spontaneous abortion/pregnancy loss at 5 weeks of gestation or later, but before the 20^{th} week of gestation, clinically detectable via sonography.
SAB/Chemical=Pregnancy loss that occurs prior to the 5th week of gestation, at which time human Chorionic Gonadotropin can measured in the maternal bloodstream but the timing is too early to sonographically detect a gestational sac.

In contrast to measurement of PAPP-A in vitro at the level of the human ovary, specifically in granulosa cells, theca cells (Conover et al, 2001; Spicer et al, 2004), follicular fluid (Botkjaer et al, 2015; Jepsen et al, 2016), and in cumulus granulosa cell masses (Kordus et al, 2019), as well as downstream in trophoblastic cells, here PAPP-A expression was observed in vitro at the human blastocyst stage. The presence of immunoreactive PAPP-A has been demonstrated in culture medium conditioned by human ovarian granulosa cells (Conover et al, 1999). PAPP-A was found to be expressed in follicular fluid, with immunostaining having shown PAPP-A localized to the theca cell layer in small antral follicles of 4-6 mm diameter, with PAPP-A expression shifting inward to the granulosa cell layer as follicles mature in size and become pre-ovulatory (Botkjaer et al, 2015). In an investigation of the potential source of PAPP-A production in pregnancy, an in vitro study in 2003 reported the expression of PAPP-A mRNA in total placental extracts, and PAPP-A protein was detected in the cytoplasm of cytotrophoblast cells as well as syncytiotrophoblast cells, with greater expression with the latter cell type's formation (Guibourdenche et al, 2003).

The present study included analyzing frozen euploid blastocyst stage embryos that had been vitrified after trophectoderm biopsy, which can be considered a strength of the study due to the ability to follow the clinical results of euploid blastocyst transfer.

The expression of PAPP-A was observed in the BFCM of preimplantation blastocyst stage embryos in vitro. Larger prospective studies can be performed and are underway regarding PAPP-A expression at the level of human blastocyst stage embryo and maternal/fetal pregnancy outcomes of trophectoderm-tested euploid blastocyst transfer. It is anticipated that additional data showing that PAPP-A in BFCM and/or biopsied embryonic cells can predict adverse obstetric outcomes. These approaches may be used in the process of embryo selection, in addition to euploid status. In this way, these methods may be used to optimize or improve the potential or probability of live birth for IVF patients.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amor et al., Hum Reprod.; 24(6):1330-8, 2009.
Antsaklis et al., Cureus., 17; 11(6):e4912. 2019.
Bøtkjær et al., Fertil Steril.; 104(5):1294-301.e1, 2015.
Bracewell-Milnes et al., Hum Reprod Update; 23(6):723-736, 2017.
Cavoretto et al., Eur J Obstet Gynecol Reprod Biol.; 198:50-55, 2016.
Chen et al., Brain Research Protocols: 4(2), p. 132-139, 1999.
Chosed et al., Integr Mol Med, vol: 6, p. 1-4, 2019.
Choux et al., Reprod Biomed Online.; 38(4):538-548, 2019.
Conover et al., Endocrinology; 142(5):2155-8, 2001.
Conover et al., J Clin Endocrinol Metab.; 84(12):4742-5, 1999.
Dunne et al., J. Obstet Gynaecol Can.; 39(10):870-879, 2017.
Gardner et al., Fertil Steril.; 73(6):1155-8, 2000.
Giorgetti et al., Hum Reprod.; 28(10):2636-42, 2013.
Gjerris et al., Hum Reprod Update; 18(4):350-9, 2012.
Gjerris et al., Ultrasound Obstet Gynecol.; 33(1):8-17, 2009.
Guibourdenche et al., Placenta., 24(5):532-9, 2003.
Huggett, et al. Genes Immun.; 6(4):279-84, 2005.
Hui et al., Prenat Diagn.; 25(5):390-3, 2005.
Huynh et al., Can Fam Physician.; 60(10):899-903, 2014.
Jepsen et al., Hum Reprod.; 31(4):866-74, 2016.
Kaijomaa et al., Prenat Diagn.; 36(12):1115-1120., 2016.
Kordus et al., J Assist Reprod Genet.; 36(7):1457-1469, 2019.
Kuznyetsov et al., PLoS One.; 13(5):e0197262, 2018.
Lal et al., Front Cell Dev Biol.; 8:78, 2020.
Li et al. Scientific Reports, volume 8, Article number: 9275, 2018.
Liao et al., Hum Reprod.; 16(7):1501-4, 2001.
Lin T M, et al., J Clin Invest.; 54(3):576-82, 1974.
Magli et al., Fertil Steril.; 105(3):676-683.e5., 2016
Matilainen et al., Fertil Steril.; 95(1):378-81, 2011.
Mortazavi et al, Nat Methods, 5: 621-628, 2008.
Neti et al., Cell Calcium, 55(1): 24-37, 2014.
Pérennec et al., Eur J Obstet Gynecol Reprod Biol.; 258:63-69, 2020.
Poli et al., EMBO Mol Med.; 7(11):1465-79, 2015.
Rule et al., J Assist Reprod Genet.; 35(8):1497-1501, 2018.
Scott et al., Fertility & Sterility 100, 624, 2013.
Scott F et al. J Obstet Gynaecol.; 49(3):258-62, 2009.
Spencer et al., Prenat Diagn.; 28(11):1029-36, 2008.
Spicer, Biol Reprod.; 70(5):1223-30., May 2004
Stigliani et al., Molecular Human Reproduction 20, 1238-1246, 2014.
Takabatake et al., 2016.
Tedeschi et al., J Assist Reprod Genet.; 34(2):225-238, 2017.
Trapnell et al., Nat Biotechnol.; 28: 511-515, 2010.
Tul and Novak-Antolic, Prenat Diagn.; 26(13):1206-11, 2006.
Vera-Rodriguez et al., Human Reproduction, Vol. 33, No. 4 pp. 745-756, 2018.
Wang et al., Arch Gynecol Obstet. 2021.
Xu et al., PNAS|vol. 113|no. 42|11907-11912, 2016.
Yaron et al., Prenat Diagn.; 22(9):778-82. September 2002.

What is claimed is:

1. An in vitro method of screening an embryo, comprising:
(i) obtaining in vitro an embryo at the blastocyst stage of development, and
(ii) measuring the expression of pregnancy-associated plasma protein-A (PAPPA) in one or more embryo cells, in the blastocoel fluid, and/or in embryo culture fluid that has been used to store or culture the embryo;
wherein a decreased expression of PAPPA below a control level indicates an increased risk of an adverse obstetric outcome if the embryo is implanted into a mammalian subject.

2. The method of claim 1, wherein the expression of PAPPA is measured in the blastocoel fluid.

3. The method of claim 1, wherein the expression of PAPPA is measured in the embryo culture fluid.

4. The method of claim 1, wherein the expression of PAPPA is measured in one or more embryo cells.

5. The method of claim 4, wherein the one or more embryo cells comprise or consist of one or more trophoblast or trophectoderm cells.

6. The method of claim 5, wherein the one or more trophoblast cells comprise or consist of syncytiotrophoblast cells.

7. The method of claim 4, wherein the one or more embryo cells comprise or consist of one or more inner cell mass cells.

8. The method of any one of claims 1-5, wherein the adverse obstetric outcome is abnormal placentation, spontaneous miscarriage, small for gestational age (SGA), intrauterine growth restriction (IUGR), intrauterine fetal demise (IUFD), or pre-eclampsia.

9. The method of any one of claims 1-8, wherein, the measuring is performed via detecting or measuring mRNA that encodes PAPPA.

10. The method of claim 9, wherein the measuring is performed via Northern blot analysis, nuclease protection assays (NPA), in situ hybridization, reverse transcription-polymerase chain reaction (RT-PCR), or next-generation mRNA sequencing (mRNA-Seq).

11. The method of claim 10, wherein the reverse transcription-polymerase chain reaction (RT-PCR) is further defined as a reverse transcription quantitative PCR (RT-qPCR).

12. The method of any one of claims 1-8, wherein, the measuring is performed via detecting or measuring PAPPA protein is a Western blot, high-performance liquid chromatography (HPLC), liquid chromatography-mass spectrometry (LC/MS), enzyme-linked immunosorbent assay (ELISA), protein immunostaining, or electrochemiluminescence immunoassay (ECLIA).

13. The method of claim 12, wherein the measuring is performed via ELISA.

14. The method of any of claims 1-13, wherein the method further comprises testing the embryo for aneuploidies (PGT-A).

15. The method of any one of claims 1-13, wherein the method further comprises selecting an embryo for implantation into the mammalian subject.

16. The method of any one of claims 1-15, wherein the embryo is implanted into the mammalian subject as part of an in vitro fertilization (IVF) method.

17. The method of claim 16, wherein the embryo has been generated from an oocyte obtained from a donor.

18. The method of claim 17, wherein the donor is the mammalian subject.

19. The method of claim 17, wherein the donor is a first human subject, and wherein the mammalian subject is a second human subject.

20. The method of any one of claims 1-18, wherein the mammalian subject is a cow, sheep, horse, dog, cat, lion, panther, ferret, goat, or pig.

21. The method of claim 20, wherein the mammalian subject is a domesticated animal.

22. The method of claim 20, wherein the mammalian subject is an endangered species.

23. The method of any one of claims 1-18, wherein the mammalian subject is a human.

24. The method of any one of claims 1-23, wherein the method further comprises performing additional genetic testing.

25. The method of claim 24, wherein the additional genetic testing comprises or consists of testing for the presence of a genetic disease in the embryo.

26. The method of claim 25, wherein the embryo is further defined as a human embryo, and wherein the genetic disease is Huntington's disease, sickle cell anemia, muscular dystrophy, cystic fibrosis (CF), a BRCA1 mutation, a BRCA2 mutation, fragile-X syndrome, or Tay-Sachs disease.


SEQUENCE LISTING (2 sequences)

SEQ ID NO: 1 - DNA, 24 bp, Artificial Sequence
  Synthetic primer
gtcatctttgcctggaagggagaa

SEQ ID NO: 2 - DNA, 24 bp, Artificial Sequence
  Synthetic primer
agggctgttcaacatcaggatgac